(19) United States
(12) Patent Application Publication
(10) Pub. No.: US 2020/0121241 A1
(43) Pub. Date: Apr. 23, 2020

(54) Breast Sense Feeding Monitor

(71) Applicant: May & Meadow, Inc., Redwood City, CA (US)

(72) Inventors: Hooman Hafezi, Redwood City, CA (US); Mary Judith Insun Feezer, Redwood City, CA (US); Douglas Alan Webb, Los Altos, CA (US)

(21) Appl. No.: 16/332,589

(22) PCT Filed: Sep. 13, 2017
(86) PCT No.: PCT/US2017/051419

§ 371 Date: Mar. 12, 2019

Related U.S. Application Data
(60) Provisional application No. 62/481,572, filed on Apr. 4, 2017.
(60) Provisional application No. 62/393,673, filed on Sep. 13, 2016.

(51) Int. Cl. A61B 5/00; A61B 5/053; A61B 5/107

(52) U.S. Cl. CPC A61B 5/4288; A61B 2560/0223; A61B 5/7435; A61B 5/4205; A61B 5/7475; A61B 5/0022; A61B 5/7278; A61B 5/1077; A61B 5/4312; A61B 5/7465; A61B 5/7246; A61B 5/486; A61B 5/6823; A61B 5/6832; A61B 2562/0261; A61B 2562/164; A61B 2503/04; A61B 2562/06; A61B 2562/04; A61B 2562/0217; A61B 5/053

(57) ABSTRACT

The Breast Sense Feeding Monitor provides real-time measurement of breastfeeding metrics including milk volume and infant suck and swallow characteristics over multiple feedings. The wearable design lends itself to versions suitable for both home and in-clinic use.

### CLAIM OF BENEFIT OF PRIOR-FILED APPLICATIONS

This patent application claims the benefit of U.S. Provisional Application Ser. No. 62/393,673 entitled “Device for Assessment of Infant Breastfeeding and Bottle Feeding” and filed on September 13, 2016 and U.S. Provisional Application Ser. No. 62/481,572 entitled “Patch for assessing breastfeeding milk supply” and filed on Apr. 4, 2017, under 35 U.S.C. § 119, which are hereby incorporated by reference in their entirety.

### BACKGROUND

An infant's ability to feed successfully is critical to their development. For newborns, especially those born prematurely, the ability to assess feeding is often critical to the child's care.

The adage “breast is best” has gained prominence both with clinicians and in the community generally. Breast milk is known to be the ideal food for babies nutritionally and to avoid colic, a serious problem for some infants. Often the antibodies a mother conveys to her child through breastmilk protect the child from disease, or mitigate illness when it occurs. Breast feeding also helps the mother and child bond emotionally.

Successful breast feeding in developing countries it particularly critical to a baby's wellbeing. With limited medical care available, vulnerable newborns and infants often suffer tragically high fatality rates. Breast milk can mitigate this serous risk, both through ideal nutrition that is easily absorb by the baby, and protective antibodies. Also, breast feeding's hormonal effects on the mother naturally providing broader spacing in her pregnancies, even without contraception. This is an important factor in both maternal and child health.

Additionally, in developing countries, baby formula is relatively expensive and of limited availability. If formula is resorted to early in a child's development, it is unlikely they will return to breast feeding. Worse, because of the cost and lack of a reliable supply chain for baby formula, children in developing countries are often have limited access to or are denied even this less optimal source of critical nutrition

Thus, in both developing and developed countries, information that encourages and enables breast feeding is of prime importance to the health and wellbeing of babies. Specifically, concrete feedback that allows better breast feeding and assures the mother and father that their child is receiving adequate nutrition from breast milk can encourage and reinforce successful breast feeding.

While the amount of milk taken by babies can be readily determined with formula feeding, the amount of breast milk consumed by a baby is often an unknown quantity. Unfortunately, the concern that their baby may not be feeding enough at the breast for optimal growth often causes mothers to abandon breast feeding in favor of formula feeding. While this suboptimal nutritional source is a disadvantage to babies in developed countries, resorting to formula feeding in developing countries can have tragic consequences in the resulting morbidity and mortality of young babies.

Some basic approaches to determine how much breast milk a baby receives have included weighing a baby before and after a feeding, or weighing their diapers to determine how much fluid and solid matter has been taken in from the breast milk. However, these are cumbersome and inexact methods, and so are rarely used on an ongoing basis. For premature infants and newborns receiving colostrum from their mothers, these methods are not practically applicable to the small volume of nutrition being received.

Scientists have responded to these needs of babies, parents and clinicians for information on breast feeding by developing devices which can provide some insight into a baby's ability to effectively nurse and receive breast milk. By example, Gurtwein teaches the weighing of the mother's breast before and after feeding her baby to estimate how much milk the child received, U.S. Pat. No. 9,211,366 B1 issued Dec. 15, 2015. Larsson teaches a ridged breast shield that uses electric resistance measurements to estimate how much milk a mother produces and the baby ingests U.S. Patent Application 2005/008035 A1, published Apr. 14, 2005. Kapon et al teach a device to assess the volume of milk cells with capacitance measurements of the breast before and after feeding. U.S. Pat. No. 9,155,488 B2 issued Oct. 13, 2015.

Currently available breast milk assessment devices can provide some information on the milk production and child feeding, typically for a single point in time. Unfortunately, these readings often do not accurately reflect the overall nutrition being provided to the infant. Also, these devices are more suited to a clinical setting, and so cannot practically provide important information to parents when the baby comes home. Information on home feedings is particularly useful, as it reflects the day to day nutrition of the baby, and gives parents ongoing feedback that their child is nursing successfully.

With the advent of personal electronic devices, and the movement for personalized medicine, such devices as Fitbit have put some of the power of clinical tests into home use, to good effect. However, these capabilities have not yet been put into the hands of parents wanting to assess their baby's ability to feed from their mother's breast.

It would be an important advancement if calculation of breastmilk provided to a baby could be provided on an ongoing basis in real time, both in home and clinical settings. This innovation would be especially valuable if it provided biofeedback to coach mothers and lactation consultants on optimal nursing techniques.

### SUMMARY

The breast sense feeding monitor provides ongoing, real time data of breast milk consumption by a nursing baby. This new system is an engineering breakthrough to meeting both the needs of parents and clinicians as it can be used both in clinical and home settings. In developing countries, breast sense feeding monitor has the potential to assure better health of babies and save the lives of infants.

To accomplish these unique capabilities, the breast sense feeding monitor system combines an impedance sensor circuit with a strain gauge sensor circuit to achieve an unprecedented flexible, robust and portable device configuration. This allows multiple measurements that are then averaged to produce an accurate picture of a baby's feeding. This innovation delivers personalized medicine results for breast feeding in both a home and clinical setting. It is a long-needed tool for optimizing breast feeding outcomes.

### Ease of Use

The small, flexible form factor of the breast sense feeding monitor sensor patch enables it to be applied comfortably and conformably to the breast of a breastfeeding mother. This important advancement allows comfortable wear for 12 hours or more, allowing multiple feedings to be measured continually and over time. The resulting large and comprehensive data set provides a very accurate reading of the baby's feeding habits and capabilities.

Moreover, the simplicity of the breast sense feeding monitor design as compared to previously available systems allows the readings to be taken in the natural setting of a home feeding. This provides a more realistic determination of the baby's feeding patterns and the amount of milk the baby is receiving.

Ease of use and the ability to easily take measurements over multiple feedings sessions is very important for at home use by mothers and babies because an infant's feeding behavior, including appetite, changes substantially from feed to feed. Therefore, a highly precise but cumbersome measurement of feeding characteristics and milk intake in a single feeding is of low value, since variations in appetite and infant alertness can result in 2× or more difference in milk intake from feed to feed. Conversely, a wearable device that provides great ease of use over multiple feedings at the expense of some accuracy in a single measurement is ideal for these mothers and babies. Ease of use includes single handed and robust operation and zero or minimal effort required from a mother to maintain or calibrate the system.

The breast sense feeding monitor achieves its unprecedented functionality through several key innovations. The flexible sensor patch is achievable through optimization of its sensing components. The impedance sensors in the flexible sensor patch produce key data as to the content of milk in the breast. The strain gauge sensors in the flexible sensor patch provide data that is synergistic to the impedance data. The result is a final report to mothers, family and clinicians that, for the first time, accurately reflect a baby's nursing ability and milk intake.

The breast sense feeding monitor systems e-data capabilities enables, for the first time, remote nursing coaching by lactation specialists. It even provides the opportunity for automated biofeedback and lactation coaching to the mother. The e-data feature also provides pediatricians and nurse practitioners remote access to key data on babies' health and development.

### Flexible Sensing Patch

The flexible sensing patch of the breast sense feeding monitor uniquely conforms to breast. As explained in more complete detail below, the fully integrated patch is provided with four or more electrodes. In the basic version of the breast sense feeding monitor, the electrodes are provided linearly in pairs, with soft fabric in between the electrodes. However, there are more complexed and nuanced configurations provided with advantages in certain applications.

The electrode measurement unit is designed to keep the electrode sensing patch extremely light. To assure that the breast sense feeding monitor system is suitable for home use, a single button can be provided that allows wakeup for the monitor with unambiguous tap pattern, and then beeps to acknowledge it is recording.

The sensing patch length can be designed to balance comfort with functionality. Typically, the sensing patch has a form factor similar to a bandage. An even shorter version optimizes comfort. However, in some embodiments of the breast sense feeding monitor where sensitivity is critical, such as a clinical setting, the sensing patch can extend from the mother's sternum to her rib cage.

The flexible sensing patch design provides opportunities for a variety of sensor placements and configurations. By example, sensors can be connected to one another via wire or they may be wireless sensors. The latter allows communication with a mobile phone or other base unit.

When sensors are in different locations, their signals must be aligned or coordinated in time. When the sensors are wired, this is accomplished by the analog signals being both fed to the same process before the data is digitized and wirelessly transmitted to the mobile phone.

When the sensors are both wireless, a useful configuration is that one sensor send its signal to the other sensor, where the signals are combined and a time stamp is applied, as opposed to both devices communicating to the cell phone. This is important to avoid latency. Latency occurs when one or more a wireless signal is sent to a mobile phone that is handling multiple operations at the same time. When the signals arrive, there may be a delay or latency in processing the signal if the phone is in the middle of other operations.

### Strain Gauge Sensor

The strain gauge sensor of the breast sense feeding monitor, in concert with the impedance sensors, provides unprecedented capabilities to measure breast milk consumption by babies. As described in more detail below, the strain gauge sensor corrects for distortions caused by movement in the mother's chest, such as from breathing, laughing, or coughing. The strain gauge sensor can also correct for other sources of Breast distortion, such as the baby squeezing or swatting the breast. These factors can badly confound the accuracy of data in currently available systems.

These problems of breast distortion during testing have been remarkably ameliorated by the combined use of impedance and strain gauge sensors in the breast sense feeding monitor. The use of the strain gauge sensor in the present invention allows a comfortable form factor for the sensor patch. It also allows free movement of mother and baby, and so provides a much more natural feeding position. This advantage encouraging long-term use of the sensor, providing much more accurate readings over time. Additionally, these readings much better reflect the actual feeding habits of the baby than those taken at only one time point.

Currently available breast feeding monitors have limited sensitivity because the electric signal detected on the breast is sensitive not only to milk content, but to deformations of the breast tissue and whether an infant or other object is making contact with the breast.

These events change the shape and size of the tissue volume within which the electric field is present or the position of the electrodes relative to each other. For example, if an infant touches the breast during feeding or if the mother or infant compress the breast, a large distortion in signal is observed and the measured signal no longer reflects milk content accurately.

As a result, in practical applications, the electrodes in currently available systems must be on a rigid support structure to ensure constant spacing and curvature relative to each other. Furthermore, the mother must be motionless and in a consistent position in order to get consistent results and allow use of the calibration step. These prior restrictions cause significant inconvenience for mothers and babies, reduce sensitivity, and prevent effective measurement of milk transfer in real time.

The strain gauge sensor of the breast sense feeding monitor also conveniently allows the calculation of breast curvature. When taken together with the impedance data, the strain gauge data is used to calculate volume by correcting measurements to better reflect actual milk content.

Measurement of throat, mouth or chest movements using a strain sensor can also be used to assess and diagnose problems with coordination of swallowing, breathing, and sucking. A piezoelectric strain gauge can be used to assess these movements simultaneously with feeding. In combination with measurement of intra-oral pressure or flow, a strain gauge can provide diagnosis of swallowing problems that interfere with normal Suck-Swallow-Breath cycles involved in feeding. Since all three components of suck-swallow-breath must function in tandem, disorganization in any one of them can be used to quantify degree of disorganization in infants with feeding problems associated with neurological development, such as pre-term babies, or poor latch.

### Impedance Sensors

The impedance sensors of the breast sense feeding monitor collect and deliver the core data on breast milk volume to the system. The impedance measurements can be taken in a variety of ways. By example, fast measurements can be taken at a single frequency, such as 10 kHz, every 0.1 seconds, and combined with periodic measurement over 3 seconds at two or more frequencies. Simultaneous measurements of impedance with strain gauge in a fast mode, such as about 0.1 seconds, can be used to detect the baby's breathing and sucks. This data can be average over 30 seconds or a minute to detect changes in breast shape.

In some embodiments of the breast sense feeding monitor, a band of strain gauge measurements is used to reduce noise in the impedance measurements data due to breast deformation.

Determination of milk quantity fed to an infant or milk flow rate during breastfeeding can be accomplished using a bio-impedance measurement, similar to that used for body fat content measurement. A decrease in the milk/fat ratio in the breast results in an increase of the electric impedance in the breast. An applied sinusoidal or square wave current (typically <1 mA) will produce a voltage detected by electrode on the breast. The voltage will provide a direct measure of the impedance change due to milk flow. Furthermore, the detected voltage signal will exhibit a phase characteristic of the amount of conductive (milk) to nonconductive (fat) matter. This is a similar principal to that used in body fat composition analyzers (e.g. the Omron HBF 306C system). Typical frequencies are in the range of 1 kHz to 300 kHz. Typically, 2 to 4 electrodes are applied to the breast in suitable locations. The electrodes may be similar to those for an EKG measurement (gel electrodes), applied to three locations around the breast, or to the breast and back of the mother. Alternatively, at least one of the electrodes may be a microneedle that penetrates the top skin layer. This configuration is attractive because it removes the contribution of galvanic skin conductance from the measurement.

### Electrode Design

Breast sense feeding monitor can utilize a number of sense and drive electrode designs. Standard EKG style electrodes can be effectively employed. However, annular electrodes have advantages in capturing data on the entire tissue of the breast. Annular electrodes also allows multiple electrode mapping if there are multiple milk annuli. This feature is shown in more details, below.

Microneedles used as the interface between the electrode and the breast allows measurement beneath skin. This choice in electrode design can limit or eliminate electrode-skin resistance problems in testing.

Multi-electrodes provide better sensitivity in data collection than single electrodes. It is advantageous to select the electrode that gives largest change for capacitance. The system interpolate electrode readings to get highest change data, providing breast volume and mapping the breast.

The electrodes can sense at various frequencies. By example, they can sense at 1-300 kHz, specifically at 1-100 kHz, and most specifically at 5 to 50 KHz. Sampling data at a single frequency is simplest, and has the advantage of the lowest power consumption, but less reliable.

Other techniques to improve raw data based on various frequencies can be used to provide greater accuracy. By example, data can be taken at two frequencies, and if they agree, the data is confirmed. If they disagree, the measurement is repeated. Three or more frequencies can be tested. If two agree, that measurement is used; if they do not agree, the measurement is repeated. These approaches are typically automated in the system.

### Universal Calibration

An important innovation unique to the breast sense feeding monitor is Universal calibration. This allows a mother to use the breast sense feeding monitor immediately out of the box without the need for the currently required lengthy individualized calibration procedures. This makes the breast sense feeding monitor ideal for use as a consumer product. For a clinical setting, more customized calibration of the breast sense feeding monitor is provided with manual expression of milk.

Currently available systems typically require a calibration step, sometimes termed a “feeding history”, to convert a signal to milk volume. This necessary calibration function depends on breast size and location of milk in the breast relative to electrodes. Combining the strain gauge and impedance sensor allows the breast sense feeding monitor to eliminate this step in favor of a universal calibration. This makes the system much more useable for home applications, providing key breastfeeding data essentially “out of the box”

### Suck and Swallow Detection

While other researchers have suggested detecting and counting baby swallows to obtain milk volume, detecting both sucks and swallows and using their ratio as a measure of milk transfer rate is a unique capability of the breast sense feeding monitor. Simultaneously detecting the resistance or capacitance of the breast (for milk volume) as well as sucks and swallows is also unique because detecting sucks and swallows is useful by itself, independent of detecting milk volume, for assessing infant suck disorganization and tracking neurological development in premature infants or other neurologically impaired infants.

### Rate of Milk Transfer

One application of detecting sucks and swallows is to provide a way to assess the rate of milk transfer by accurately counting the number of sucks and swallows. Babies typically suckle on a breast until enough milk has been extracted for a full swallow or gulp. When milk flow into the baby's mouth is relatively slow, a baby may suckle 5-10 times in between swallows. When milk flow is high, the number of sucks in between swallows is lower, such as 1-2 sucks for each swallow. Therefore, the number of sucks per swallow is a good measure of the rate at which breast milk is flowing into the baby's mouth. Furthermore, the number of swallows in a given time period combined with an average swallow volume can provide a measure of a baby's intake.

### Neonate Testing

This kind of detection is useful in assessment of colostrum volume, low milk volumes, or the progression in milk production immediately after birth and during the first 1-2 days after birth. After birth, an infant's suckling movement promotes the production of hormones that initiate milk production. During the first 1-2 days after birth, the breast initially produces a small amount fluid known as colostrum. Colostrum has a thicker consistency and lower volume than the breast milk generated once milk production has fully commenced (past the onset of lactogenesis II). If the volume of colostrum is too low, it may be difficult to measure it with precision using changes in breast impedance alone. However, the suck-to-swallow ratio and number of swallows can be tracked to measure the gradual increase in milk production. Eventually, once the milk volume is sufficiently high, the impedance sensor may be utilized.

### Feeding Ability

A second application for detecting sucks and swallows is to assess feeding ability in high-risk infants with medical conditions that can affect feeding ability. Infants with neurological problems, such as premature infants, often have difficulties in coordinating the suck-swallow-breath motions required for successful feeding. These infant will typically sucks a few times, but are unable to sustain a succession of sucks for effective feeding. Furthermore, the number of sucks in between swallows can indicate the infant's suck strength, a useful metric for monitoring progress in infant's recovering from trauma, such as cardiovascular defects and surgery.

In certain situations, a more precise measurement of an infant's sucks and swallows are desired than would be possible with a sensor located on the mother's breast. In these applications, a smaller “Baby Sense” patch can be placed on the infant's chin or throat. The intent is to detect movements that correspond to sucks and swallow and potentially breathing in a location on the infant's body that provides better sensitivity than a patch on the mother's breast.

This “Baby Sense” patch can be used in conjunction with the “Breast Sense” patch on the mother's breast. In some instances, the “Baby Sense” patch may also be used separately on its own. It would contain a strain gauge or an impedance sensor, or both.

### BRIEF DESCRIPTION OF THE DRAWINGS

FIG. 1 is a diagrammatic view of the Breast Sense Feeding Monitor system, showing the components in the first and second layers of the wearable patch.

FIG. 2A provides a broad view of the Breast Sense Feeding Monitor system in use by mother

FIG. 2B shows a larger view wearable patch of the Breast Sense Feeding Monitor system

FIG. 2C shows a larger, more detailed view of mobile phone and GUI of the Breast Sense Feeding Monitor system

FIG. 3A shows the basic patch design for the Breast Sense Feeding Monitor system

FIG. 3B illustrates a distributed patch design for the Breast Sense Feeding Monitor system

FIG. 3C is a hybrid configuration where the sensing second layer is divided into two pieces.

FIG. 3D is a detailed view of FIG. 3C

FIG. 4A shows a basic arrangement for the impedance sensing electrodes,

FIG. 4B shows a design with more than four impedance sensing electrodes,

FIG. 4C is a cross-section of typical gel electrode,

FIG. 4D illustrates a top view of an alternative electrode configuration,

FIG. 4E illustrates bottom view of the patch with alternate electrode configuration

FIG. 4F illustrates a top view of the wearable patch with feature for reproducible positioning on the breast

FIG. 5A illustrates the electrodes to be applied to the breast prior to measurement,

FIG. 5B shows what the patch after the measurement is complete

FIG. 6 shows the microneedle electrode design,

FIG. 7A shows a strain gauge sensor bending measurement in one direction,

FIG. 7B shows two strain gauge sensor in different directions,

FIG. 8A is a graph of the output of an impedance sensor for a typical feeding session

FIG. 8B is a graph of the strain gauge output

FIG. 8C data from impedance and strain sensors and the combinations of the data to reduce noise

FIG. 8D shows universal calibration curve

FIG. 9A shows the operation of the Breast Sense Feeding Monitor system at different frequencies,

FIG. 9B shows showing detection of suck during the single frequency part of the measurement.

FIG. 10 shows a patch configured with an additional impedance or strain sensing location in the latch area to detect sucks and swallows

FIG. 11A shows the simultaneous use of Breast Sense Patch and Baby Patch on the baby abd breast

FIG. 11B shows real time milk volume output from the Breast Sense Patch

FIG. 11C shows detection of sucks and swallows at the additional sensing location

### DETAILED DESCRIPTION

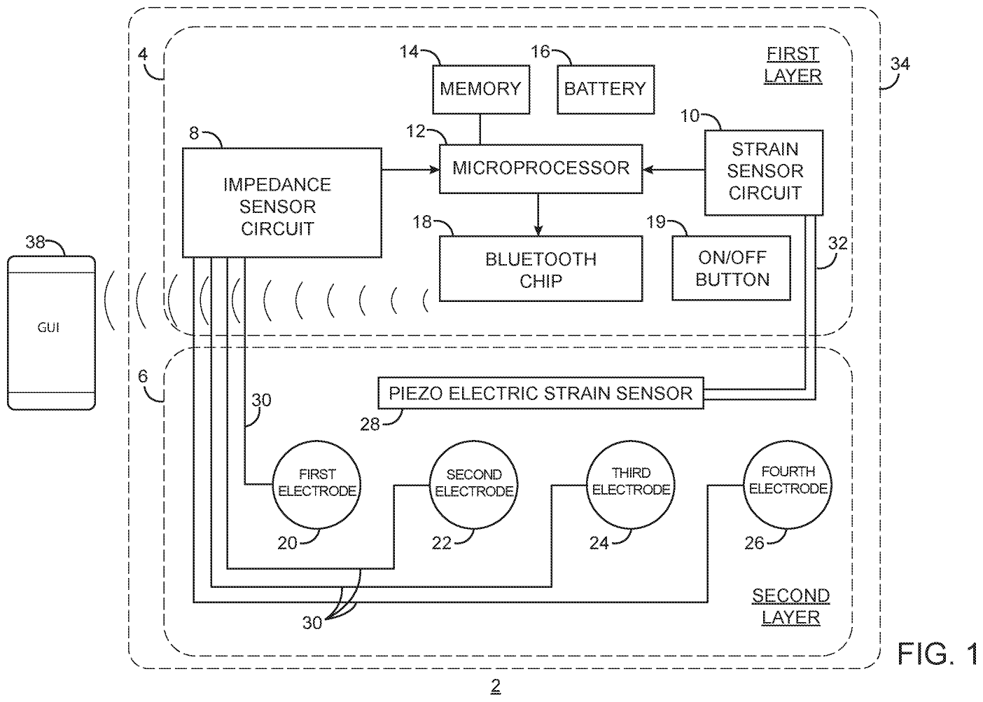

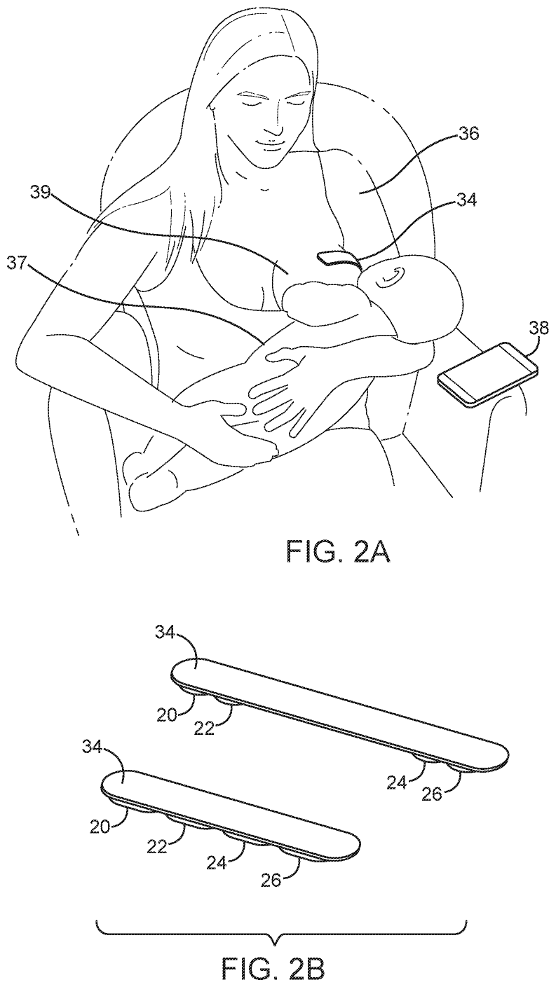

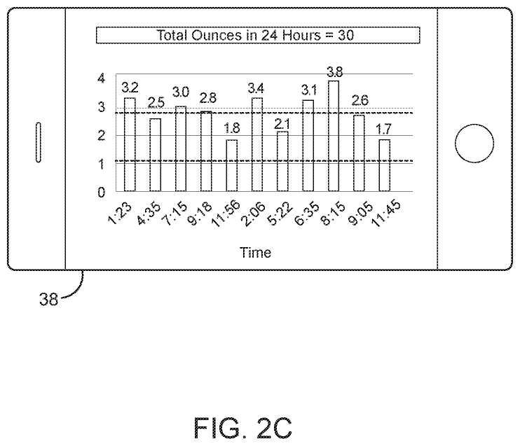

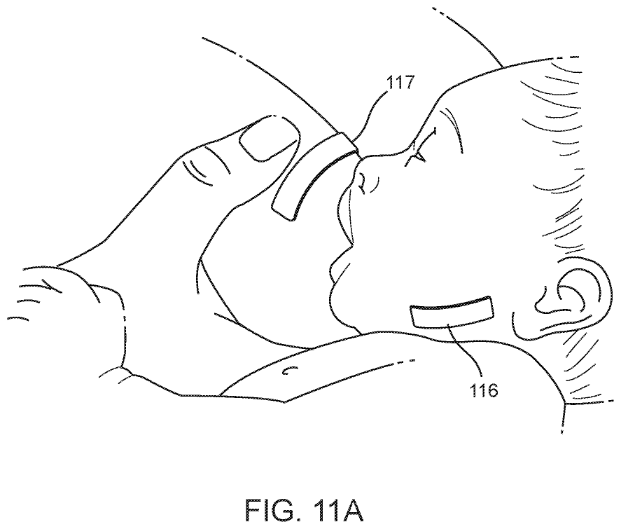

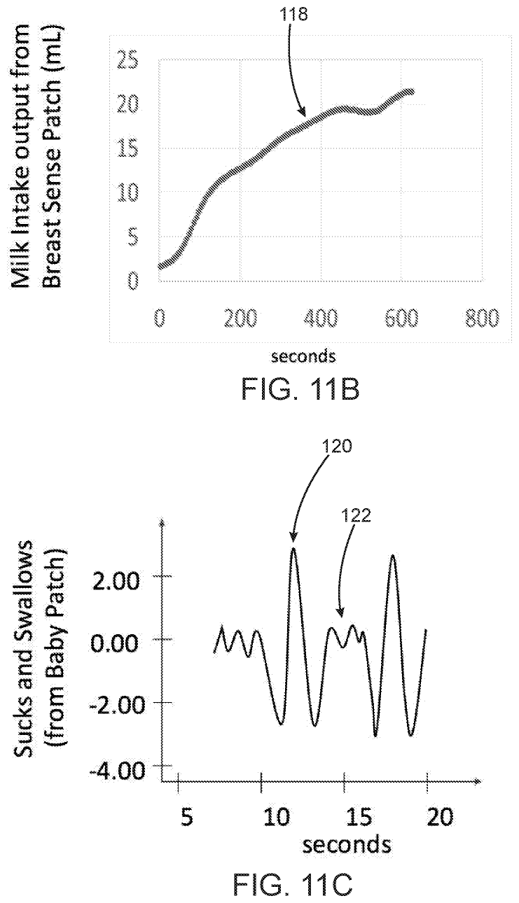

The Breast Sense Feeding Monitor system achieves its unique advantages and capabilities through the synergisms between its key components. A central feature of the Breast Sense Feeding Monitor system is wearable electronic patch, the Breast Sense Patch. The Breast Sense Patch detects changes in the breast's milk content as well as key parameters related to an infant's suck and swallow pattern. This information is communicated wirelessly to a mobile phone or other user interface. While collecting data, the Breast Sense Patch, is placed on the mother's breast during one or more breastfeeding sessions. FIG. 1 shows one configuration of the Breast Sense Patch and its internal components as part of the Breast Sense Feeding Monitor system. FIG. 2A shows the Breast Sense Patch 34 and mobile phone 34 during use by a breastfeeding mother and baby. An optional additional patch, the Baby Patch, can be placed on the infant to collect additional data in certain cases and is described later in FIG. 11.

The components of the Breast Sense Feeding Monitor system can be designed in a variety of configurations. These configurations are selected to best suit a particular application. One such configuration of the Breast Sense Feeding Monitor components is shown diagrammatically as several blocks in FIG. 1.

As shown in FIG. 1, the Breast Sense Feeding Monitor system 2 includes Breast Sense Patch 34 which is composed of two layers of the system's components. These components work together to provide real-time sensing and reporting of the amount and rate that a baby receives breast milk from their mother. In some cases, additional information is obtained by the Breast Sense Feeding Monitor system, such as the baby sucking characteristics such as strength, rate, and quality.

In FIG. 1, first layer 4 is a physical region of the Breast Sense Patch 34. First layer 4 provides and supports much of the functionality the Breast Sense Feeding Patch 34. The circuitry in first layer 4 supports the sensing, calculation and reporting functions of Breast Sense Feeding Monitor 2.

Some examples of the circuitry which can be included in first layer 4 of Breast Sense Feeding 34 includes impedance sensor circuit 8, strain sensor circuit 10, and microprocessor 12. Impedance sensor circuit 8 functions to apply a sinusoidal electrical to body through 2 drive electrodes 20 and 26, as shown below, sense the resulting voltage on the body using 2 or more sense electrodes 22 and 24 as shown below, and convert the detected quantities to digital signals for processing. Typically, the impedance circuit must provide voltage sufficient to drive a current of up to of up to 1 mA RMS such as about 100 uA to 500 uA, through the breast tissue, at a frequency ranging from about 0.1 to 1 MHz, such as about 1 to 100 kHz.

In one implementation, the impedance circuit applies a voltage suitable for driving the desired current through the body, measures the resulting current flow and the voltage at the sense electrodes simultaneously, then processes the data to derive the desired output and transmit this information to the microprocessor 12. An example of an impedance sensor circuit is the Texas Instrument AFE4300 system on a chip. Alternatively, a custom circuit can be designed around a network analyzer chip such as the Analog Devices 12 bit AD5933. Other circuit designs suitable to this application will be well know to one of ordinary skill in the art.

The strain sensor circuit 10 receives sensor data on strain measurements from a sensor such as a piezoelectric strain gauge. Shown below. The sensor output is typically detected using a half or quarter bridge circuit, converts the analog signal to a digital signal, and transmits this information to microprocessor 12. In one embodiment, the TI AFE4300 System on Chip integrates both impedance sensing and strain sensing circuits into one package and can be used for both functions. Alternatively, a custom strain sense circuit is designed using an appropriate bridge circuit and differential amplifier such as the AD8220.

The communication of the impedance sensor circuit 8 and strain sensor circuit 10 to the microprocessor 12 are shown in this view by arrows indicating the direction of flow of information. While in this view of Breast Sense Feeding Monitor 2 the communication between the impedance sensor circuit 8 and strain sensor circuit 10 to the microprocessor 12 is via wires, in alternative embodiments of the Breast Sense Feeding Monitor 2 system, this communication can be accomplished wirelessly, or by integration of all three components into a single micro-circuitry chip.

Also provided in first layer 4 is optional non-volatile flash memory chip 14 and battery 16. Memory chip 14 serves to store software and settings to operate the Breast Sense Patch 14 and retain software and settings when the system is powered down Also, should there be an interruption in power or delay in transmitting the data to the mobile phone 38, the non-volatile memory can retain some or all the data collected by the Breast Sense patch as a backup.

It is useful if memory chip 14 of at least about 20 MB storage capacity and preferably at least about 40 MB storage capacity, and write speed of at least about 10 kHz. A variety of memory chips can fulfill this requirement, such as the Cypress Semiconductor S25FL256S or equivalent chips.

Battery 16 provides power to all components contained in Breast Sense Feeding Monitor 2. By way of example, the battery may be a lithium ion battery capable of providing about 3 to 3.8 V voltage and a capacity of about 120 mAh to 350 mAh, such as about 150 to 220 mAh over a discharge time of about 3 to 24 hours, such as about 5 to 10 hours, and a current of up to about 40 mA. This capacity provides total usage for at least ten 30-minu feeding sessions over the course of a day. Battery 16 may be rechargeable or non-rechargeable. Examples of non-rechargeable batteries include CR2032, R2032, CR2330, BR2330 batteries. Examples of rechargeable batteries include RDJ3032 or RDJ2440 batteries. If a rechargeable battery is used, a suitable charging circuit must be included in the battery component 16.

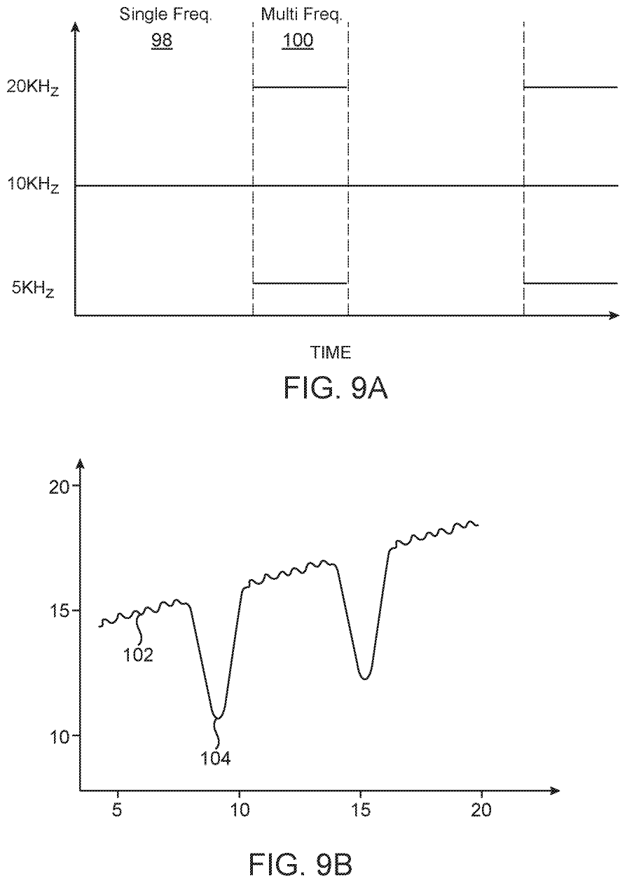

The battery component may further include power management circuitry to enable the Breast Sense Patch 34 to automatically enter a low power consumption “sleep” mode if no active feeding is occurring for a certain amount of time, such as about 2 or about 5 minutes. In sleep mode, the system may at least one sensor circuit at a low frequency to look for a signal characteristic of active feeding and “wake up” the Breast Sense Patch 34. An example of such a signal is the occurrence of high frequency, low amplitude undulations in the impedance sensor signal 102 or strain sensor signal 122 as shown later in FIG. 9B and FIG. 11C.

A Bluetooth chip 18 is provided for Breast Sense Feeding Monitor 2 for wireless transmission of data to cell phone 38. The Bluetooth chip 18 conveys key information, in a manner helpful and tailored to the user, to the cell phone 38 for communication to the user. In some embodiments of Breast Sense Feeding Monitor 2, some of the functions provided by the circuitry in first layer 4 is provided in said cell phone 38. In other embodiments of Breast Sense Feeding Monitor 2, the raw or partially processed data from the sensors in second layer 6 is transmitted to the cloud, processed, and then returned to the cell phone to be displayed to the user.

A variety of electronic components and combinations may be used to fulfill these functions. For example, the Cypress Semiconductor CYW20737 SOC and the Atmel ATBTLC1000 QFN BLE Bluetooth SoC incorporate microprocessor and Bluetooth chips into one component. The Silicon Labs EFR32BG1 chip is a microprocessor that provides at least about 20 MHZ clock speed and combines microprocessor, Bluetooth, program memory and ram, digital and analog i/o, real time clock, dc/dc converter, analog-to-digital and digital-to-analog converters, and bluetooth into one package.

First layer 4 also contains optional on/off button 19. On/off button 19 allows the user, after applying the patch, to imply hit that button before breastfeeding, and then hit it again at the end of breastfeeding. This tells the device to go back into a sleep mode. A physical button has advantage over having this function controlled by the cell phone. For instance, during operation of the Breast Sense Feeding Monitor system, most mothers are handling a baby with one hand. Thus, in some cases, scrolling through screens and otherwise working on a cell phone is less convenient than having an actual button on the patch.

A physical on/off button provides that on and every time a measurement is to be accomplished, the user simply hits go, right, and the device runs. At the end of the measurement, the user hits the button again to turn the device and recording off. In a different embodiment, each time the button is hit, the device runs and collects data for the next half-hour. Within that button you can have sort of implements to make it robust. By example, a code can be implemented “two taps means start,” and “three taps means turn off”.

Second layer 6 of Breast Sense Feeding Monitor 2 contains the impedance sensing electrodes for the impedance sensor circuit 8. The impedance sensing electrodes are first electrode 20, second electrode 22, third electrode three 24, and fourth electrode 26. In some configurations of the Breast Sense Feeding Monitor 2, there may be more or fewer of impedance sensing electrodes, but in many cases the preferred configuration is four.

The impedance sensing electrodes, first electrode 20, second electrode 22, third electrode 24, and forth electrode 26, are connect via connecting wires 30 to impedance sensor circuit 8. As described above, the impedance data for Breast Sense Feeding Monitor 2 from the impedance sensing electrodes is thus conveyed via strain sensor circuit 10 to the microprocessor 12 and therein to the user.

Similarly, the strain sensor 28, also provided in second layer 6, connects via wire 32 to the strain sensor circuit 10. For the purposes of this application, strain sensor is defined as any mechanical sensor capable of detecting a deflection, or displacement of all or part of the Breast Sense Patch, such as a piezoelectric strain gauge sensor, capacitive mesh sensor, a pressure sensor, or equivalent. That information is then combined with the strain sensor data in the microprocessor 12 to provide more compressive, synergistic data to the user then that from the impedance sensing electrodes alone. This synergism is described in greater detail elsewhere in this application.

FIG. 2A provides a broad view of the Breast Sense Feeding Monitor 2 being used by mother 36. Mother 36 applies test patch 34, which contains all the components shown in FIG. 1, above, to her breast 39 before the first feeding of baby 37. In a typical Breast Sense Feeding Monitor 2 testing situation, mother 36 leaves test patch 34 on during the entire period during and between four or more feedings. The Breast Sense Feeding Monitor 2 collects data from both sensors simultaneously over the course of those four or more feedings, combines the input from the sensors in a unique way to get a highly accurate measure of milk intake. In many cases, a number of other parameters are included in the analysis of the data. This final information is then transmit those to mobile phone 38.

FIG. 2B shows a larger view wearable patch 34 in two different possible configurations among a range of different designs appropriate to the Breast Sense Feeding Monitor 2. The hardware of the Breast Sense Patch 34 can, in one embodiment, be designed and fabricated such that first layer 4 and second layer 6 are superimposed, so that they configured top of each other inside a wearable patch cover. The Breast Sense Patch 34 is from about 3-10″ in length, more specifically about 6″-8″ in length, and most specifically about 7″ in length. The Breast Sense Patch 34 is designed to be as thin and light as possible and may have a thickness of 2 to 35 mm at it's thickest point, more specifically 2 to 15 mm, and most specifically about 10 mm.

FIG. 2C shows a larger, more detailed view of mobile phone 38. One approach to the graphic user interface is shown on the screen of mobile phone 38. However, the information can be conveyed to the mother 36 audibly as well, such as with a varying tone, or specific beeps when different points in the conveyance of the milk to the baby 37 are reached. Also, the information can be conveyed to a lactation specialist or other health care provider.

Note that while the device to provide information to the user is illustrated in this and the following figures as a smart phone, the interface can be any number of personal electronic devices, such as tablets, computers, TV screens, etc. Additionally, the user interface need not be graphic. By example, a speaker can provide audio cues, and vibration cues could also be employed.

In most applications, ease of use and comfort of the mother are far more important than the accuracy or precision of a single measurement. This is because an infant's appetite or milk intake can vary by more than a factor of 2 between feedings. Therefore, it is essential to take measurements over multiple feedings, typically about 4 to 6, to obtain a truly representative measure of an infant's feeding. Therefore, a patch that is more comfortable and can be readily worn over multiple feedings is preferable to a bulkier, less comfortable patch that may provide greater sensitivity for a single feeding session but is inconvenient to wear over multiple feedings.

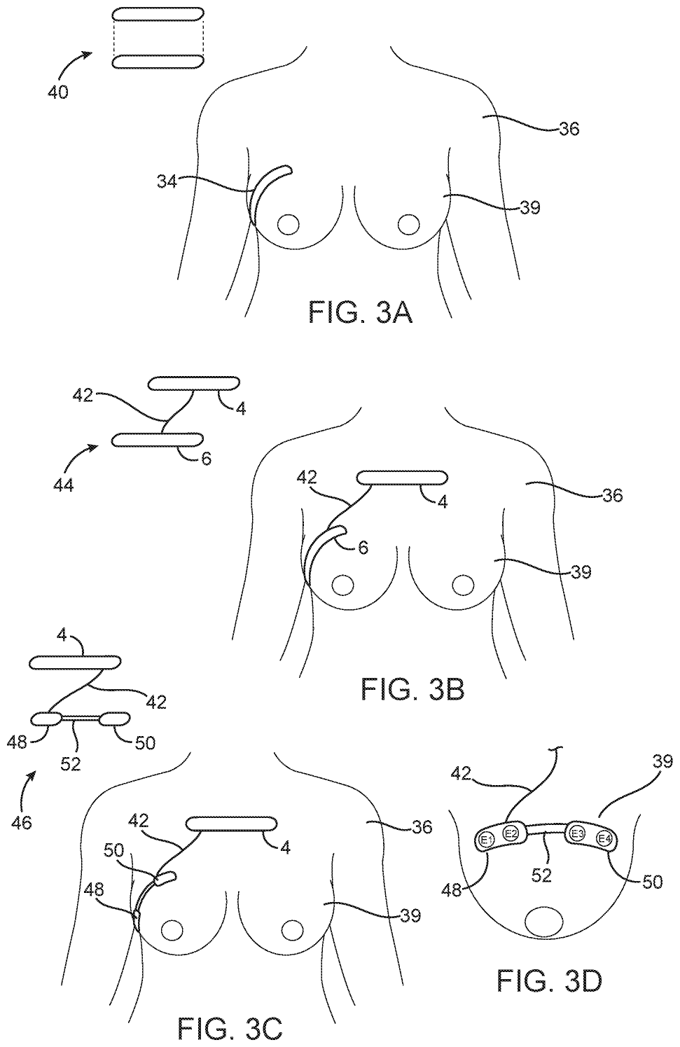

The Breast Sense Feeding Monitor system is highly adaptable to different form factors and applications, and can be designed in various configurations most suitable to a particular use. By example, clinical applications in a hospital will benefit from different designs than those used in a more consumer product application. FIGS. 3A, 3B, 3C, and 3D show alternative embodiments of the Breast Sense Feeding Monitor system 2 hardware configurations to provide different combinations of comfort and sensitivity.

In some applications, the configurations shown in FIGS. 3B, 3C, and 3D will have advantage over the basic configuration shown in FIG. 2B, exemplified in FIG. 3A. The configuration in FIG. 2B is basically two layers positioned directly on top of each other, combined into one body. In FIG. 3A, this two layer configuration 40 show as a simple graphic the configuration describe in grater internal detail in FIG. 1.

An alternative configuration as shown in FIG. 3B provides that the two layers of Breast Sense Feeding Monitor, first layer 4 and second layer 6 are configured as two separate pieces that are connected with layer connecting wire 42 to construct distributed patch design 44. In this case, first layer 4 of distributed patch design 44 can be positioned on the chest of mother 36.

Unlike the basic configuration shown in FIG. 1 and FIG. 2B, in FIG. 3B first layer 4 is its own packaged unit. First layer 4 contains the bulkiest electronic components, including the system battery. In this case, first layer 4 can, in certain embodiments of the Breast Sense Feeding Monitor system, be positioned on the mother's chest or elsewhere. Second layer 6 is separate from first layer 4. Second layer 6 contains only passive components such as electrodes and strain sensor and can be made extremely thin, flexible, and lightweight. The two are connected through layer connecting wire 42. Connecting wire 42 is basically a combination of connecting wires 30 into a wire bundle. In design 44, the light and highly conformal second layer 6 remains on the breast for multiple feedings. The bulkier layer 4 can be worn on the chest, sternum, armband or alternative location that is more comfortable or discreet. Alternatively, in additional embodiment, layer 4 can be located off the body, such as on a shelf or countertop. Layer connecting wire 42 would be a detachable wire that allows layer 4 and layer 6 to be connected when the mother and baby are about to commence a breastfeeding session and disconnected when the feeding sessions is over.

FIG. 3C shows a hybrid configuration 46 second layer 6 is divided or split into two pieces. This configuration provides even greater flexibility that the configuration shown in FIG. 3B. As a result, hybrid configuration 46 gives even more comfort in wearability of mother 36.

As in FIG. 3B, first layer 4 and second layer 6 are configured as two separate pieces that are connected with layer connecting wire 42. However, in this case second layer 6 is split into second layer part A 48 and second layer part B 50. Note that the layer connecting wire 42 can be connected to either second layer part A 48 or second layer part B 50.

In this embodiment, second layer part A 48 and second layer part B 50 each contain two electrodes. Second layer part A 48 houses impedance sensing electrodes, first electrode 20, second electrode 22. Second layer part B 50 houses impedance sensing electrodes third electrode 24, and forth electrode 26. These impedance sensing electrodes are not shown in this view.

As shown in both FIG. 3C and in further detail in FIG. 3D, second layer part A 48 and second layer part B 50 are both attached and spaced apart by strain sensor layer 52 which contains the strain sensor 28. Thus, both the strain and Impedance sensing functions are incorporated in this distributed embodiment of second layer 6 in sensor layer 52. Note that layer connecting wire 42 can be attached to either second layer part A 48 as shown in FIG. 3C or second layer part B 50 as shown in FIG. 3D

Incorporating these design elements providing additional flexibility into an optimal Breast Sense Feeding Monitor system configuration provides long-term wearability for mother 36. The distinction between the separate configuration of the layers in the hybrid configuration is simply that second layer 6 is now split into separate units. Instead of this functionality being in one piece, there is a functionally longer piece. These two design configurations are not distinct in terms of function, only in physical configuration. It is more how the elements are arranged inside the housing, because these elements are still connected in terms of communication. The difference is the amount of stiffness that is ameliorated when the bulk housing is separate out.

The hardware configuration of Breast Sense Feeding Monitor can usefully be geared toward providing maximum flexibility, and resulting increased comfort, for mother 36, This advancement allows the measurement to be done for an extended duration, giving the most complete and accurate results. With this new functionality, the measurement is not actually a single measurement of a feeding, with maximum accuracy. Rather, the Breast Sense Feeding Monitor device 2 lends itself to measuring an average of total four or more connecting feedings. That insight is the motivation for these engineering features.

The total length and overall functionality of second layer 6 or second layer part A 48 and second layer part B 50, combined, in any of these configuration is important to achieving the best possible functionality for Breast Sense Feeding Monitor. As described previously, the length of the patch of second layer 6 is often 4-8 inches.

Some of the present inventors have developed data during studies of Breast Sense Feeding Monitor system prototypes around the effect of the length of second layer 6. This length influences signal strength, and so needs to be selected appropriately. The location of the patch containing second layer 6 on the breast is important. By example, it was determined that that placing the patch 3-6 centimeters from the nipple gives the best signal strength. This appears to be true in subjects with a range breast size and shape.

Because of the ease and flexibility of the Breast Sense Feeding Monitor system, mothers will be able to modify the placement of the sensor patch to the optimal location, both to optimize sensing and comfort, on breast 39.

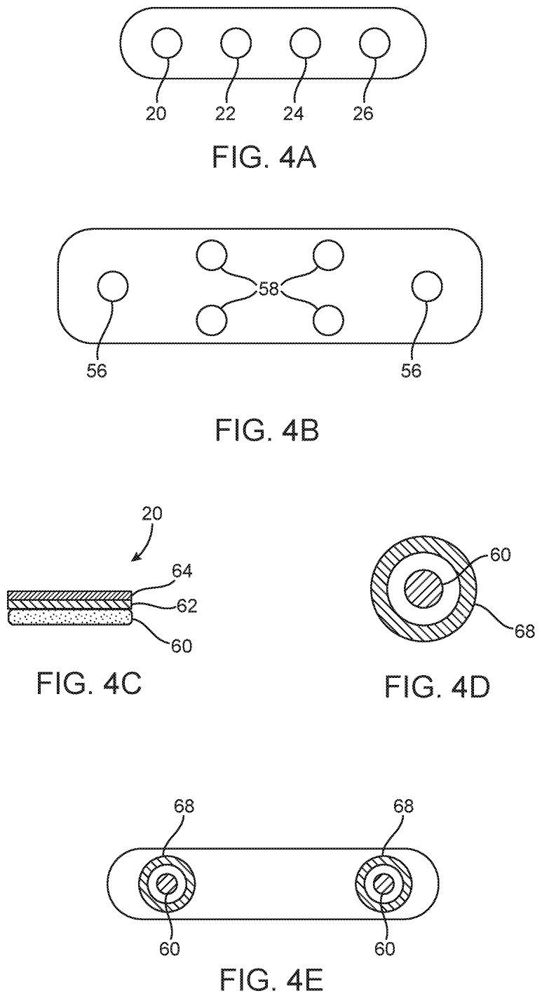

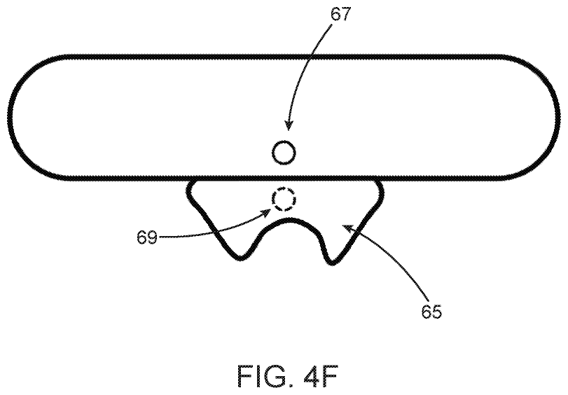

FIG. 4 shows different arrangements for electrodes for the impedance sensor, and various designs for the electrodes themselves. The most basic arrangement for the impedance sensing electrodes is shown in FIG. 4A. The impedance sensing electrodes, first electrode 20, second electrode 22, third electrode 24, and fourth electrode 26 are positioned collinearly, so that they are situated in a row within second layer 6. Two of them, first electrode 20 and fourth electrode 26, are conventionally considered the drive electrodes. These are the electrodes that are used to inject current into the body. The other two, second electrode 22 and forth electrode 24, are called the sense electrodes.

The difference between the sense electrodes and the drive electrodes is during the impedance measurement part of the device. The impedance sensor functionality involves driving a sinusoidal current through the drive electrodes in contact with the body. Then the voltage that exists in the body is measured by the Breast Sense Feeding Monitor system 2 with the sense electrodes.

Typically in impedance, two electrodes are used to inject current. Two other electrodes are used do the measurement. This is a convention, rather than a necessarily dedicated use of an electrode. Thus, the drive electrodes could be used to do sensing as well.

Multifunctional electors allow a flexible use of the Breast Sense Feeding Monitor system 2. By example, the Breast Sense Feeding Monitor system 2 can alternate between driving through first electrode 20 and fourth electrode 26, and sensing with second electrode 22 and third electrode 24. This mode of operation is in contrast to driving through electrode first electrode 20 and fourth electrode 26, and actually sensing with those same electrodes. Most typically, the Breast Sense Feeding Monitor system 2 will be driving with first electrode 20 and fourth electrode 26, and sensing with second electrode 22 and third electrode 24. The system can move fluidly between any of these modes, even very rapidly in the same session, to produce optimal functionality for the Breast Sense Feeding Monitor system 2.

FIG. 4B illustrates an alternative arrangement impedance sensing electrodes employing more than four electrodes. In this case there are six rather than four electrodes. In this embodiment of the Breast Sense Feeding Monitor system 2, drive electrodes 56 and sense electrodes as 58 are provided. Thus, in this configuration there are two more sense electrodes than show in FIG. 4A. During sensing, the drive current is still driven through electrodes 56. However, in this case it is possible to sense between different pairs of electrodes among the sense electrodes 58.

The advantage to this electrode configuration is that a potentially more actuate assessment of milk volume in the breast 39 can be provided. The milk reservoir in the breast, that is where the milk is stored in the breast, can be in different locations. This may not directly correspond to where the Breast Sense Feeding Monitor patch is applied to breast 39.

Because of natural anatomic variability, cells containing the milk may be higher or lower on different subjects. The greatest signal strength is if the sense electrodes are closest to where most of the milk reservoir are located. With multiple electrodes, there is the option of sensing different combination of electrodes and picking the one that gives the most signal.

This opportunity for optimal spacing this advancement represents is not currently available with existing system. Appreciating and accounting for the effects of nodular pooling provides the opportunity to achieve fully accurate sensing data. For this reason, a configuration such as the one above is especially useful in a clinical setting, where highly accurate data in fewer test sessions is more importee.

There are a verity of factors which can influence the optimization of data collection with the Breast Sense Feeding Monitor system. The breast changes over the course of feeding, both within feedings and over time. By example one pair of electrodes is more sensitive during the first few days of feeding after birth. Over time, the breast essentially maps itself out with the baby's changing in feeding and the changing milk consistency, content and volume. A different location for sensors may be optimal, say at week 2, 3, or 4 post-partum.

With these changes, having the multiple electrodes allows the Breast Sense Feeding Monitor system to be able to better handle and adjust to those changes, rather than simply rely on a minimum of four electrodes.

This heightened sensitivity and high accuracy will not be necessary for many applications. In those uses of the Breast Sense Feeding Monitor system 2, it may not be optimal to complicate the system, since this complexity can come with disadvantages of their own. For instance, the Breast Sense Feeding Monitor device would be bigger and, likely, less comfortable. Whether a design using just four electrodes, or one employing more is better suited to an application, will depend on the demands of the particular application and how truly accurate the results are required. In practice, some of the present inventors have found that four electrodes provide enough sensitivity for most applications.

Another advantage to having multiple electrodes in the Breast Sense Feeding Monitor system 2 is that the system can *sense* through different pairs of electrodes in the 58 group, and generally map the location of optimal sensitivity by interpolating the signal. In this manner the signal can be assessed at various locations. With sensing from different pairs of electrodes map, the less sensitive spots could be identified, narrowing down to the most sensitive spot. For instance, the most sensitive spot may be located three quarters of the way between two different pairs of electrodes. To facilitate this mapping capability, more than four electrodes can be provided under 58.

This optional mapping function allows the potential for optimization of sensing, which is particularly key in applications such as clinical settings for preterm babies, or newborns receiving colostrum from their mothers.

An important feature for the Breast Sense Feeding Monitor system 2 when used as a consumer product is its ease of use. In a home setting, the mapping system would be optional, and in many cases unnecessary to get key information. Some of the present inventors have been told by clinicians that it is preferable to have a simple, easy-to-use system for home use. However, that in a doctor's office, a more full featured system with better resolution for this relatively shorter testing period would be more appropriate.

As shown in cross-section FIG. 4C, an electrode being used in the Breast Sense Feeding Monitor system as impedance sensors is typically gel electrode 20. Gel electrode 20 is provide with gel layer 60, silver-silver-chloride layer 62 and conductive backing 64. Examples of commercially available electrode types that may be suitable are 3M 2228, Vermed A10022, and Coviden Kendall H69P neonatal gel electrodes. These gel electrodes serve to make electrical contact with the body and hold the Breast Sense Patch 34 in place. Custom made gel electrodes can be made with the desired size, shape, and adhesive strength to ensure comfort.

FIG. 4D illustrates a top view of an alternative electrode configuration. This alternative electrode design allows the drive and sense electrodes to be combined in a way that is more compact and gives better resolution. Again, there is a conductive section with a black silver-silver-chloride coating. A gel layer is provided to facilitate adhesion and conductivity.

However, in this case, a sense electrode 66 is at the center of the alternative electrode design. Sense electrode 66 has all the same layers as illustrated in FIG. 4C. But in this alternative electrode configuration, the sense electrode 66 surrounded by an annular drive electrode 68.

This kind of configuration had advantages over the basic electrode configuration illustrated in FIG. 4A. In FIG. 4A there are four distinct electrode areas. The configuration in FIG. 4C allows the combination at of combine these two and two, so you would effectively have two electrode areas.

This opportunity for different second layer 34 design made possible by the FIG. 4C electrode is show in in FIG. 4E. The patch with the alternative electrode design would look more like a two-electrode patch. However, functionally, this is equivalent to the basic four electrode patch since each electrode area has functionally two electrodes.

One advantage of the electrode configuration in FIG. 4E versus that in FIG. 4A is that because there are only two electrode areas, the whole patch can be smaller and more flexible. With four areas, the patch can be a little stiff. However, by reducing the four electrode areas to two, there is more flexibility.

Another advantage of the electrode configuration in FIG. 4E versus that in FIG. 4A is in sensing. In the case of the electrode configuration in FIG. 4E, both sense electrodes 66 and drive electrodes 68 are provided.

In FIG. 4A the drive electrodes, first electrode 20 and fourth electrode 26, are outside the sense electrodes, second electrode 22 and forth electrode 24. In 4E the sense electrodes 66 are measuring a voltage that is essentially right in the middle of the drive electrodes 68. This provides a bigger signal.

In FIG. 4A the biggest voltage difference is between the points at first electrode 20 and fourth electrode 26. When the sense electrodes are inside these points, only about half of that voltage is sensed. The voltage varying almost linearly from the points at 20 to 26, so only a portion of that is sensed. By contrast, configuration shown in FIG. 4E, results in a much larger signal, which is less prone to noise. This improves the signal-to-noise ratio is improved.

This configuration is also less sensitive to the exact location of where the milk reservoir. What happens is, if in FIG. 4A, the milk reservoir is right underneath this drive electrode, the measurement is accurate. However, if the sensing electrode is off to the side of the milk reservoir, the system will not catch the effect of that milk reservoir as well. A reduced signal strength results

However, when the sensing and driving electrodes are collocated, as in FIG. 4E, there is a more generalized sensing. This is because whatever happens between, as the current travels through the breast is going to show up in these two sense electrodes. They can't possibly be dislocated relative to the milk reservoir. The ideal situation is when those two are actually at the same location relative to the milk reservoir. That gives the biggest signal. The configuration shown in FIG. 4E can be modified to have multiple electrode areas, similar to FIG. 4B.

In summary, the various design strategies shown in configurations shown in FIG. 4A, FIG. 4B, FIG. 4C, FIG. 4D, and FIG. 4E can be generalized to the following design consideration. It is always possible add more electrodes to the Breast Sense Feeding Monitor system. However, an increased number of electrodes, while increasing sensitivity, comes at the expense of larger size and less comfort. Thus, the ordinary skilled artisan will consider design parameters for the end use to optimize effects and balance these considerations.

The Breast Sense Patch 34 may include an optional feature to enable consistent positioning of the patch relative to the nipple. FIG. 4F is a top view of a Breast Sense patch illustrating this feature. Feature 65 is a positioning flap made of fabric or plastic with a cutout for the nipple to allow the Breast Sense Patch 34 to be positioned at a precise distance from the nipple. Once the Breast Sense Patch has been positioned and attached to the breast, the flap is folded back onto the patch so that it is not interfering with the baby's latch. This is done using attachment components 67 and 69 which may be snaps, buttons, velcro patches, or other attachment mechanism.

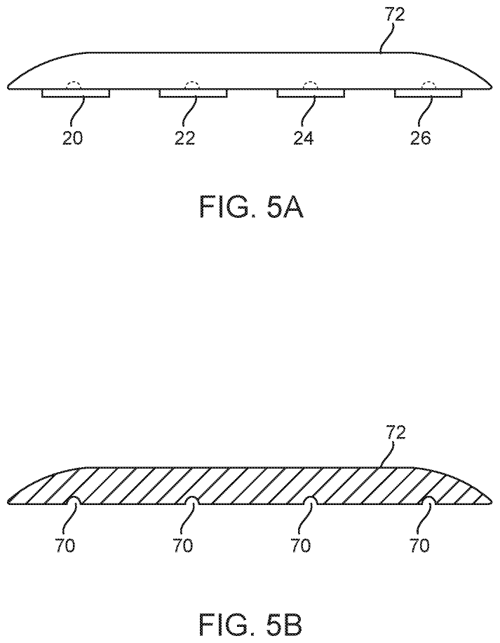

FIG. 5 illustrates that, when electrodes are described in the above figures, they are referring to removable electrodes. These are gel electrodes that adhere to the body. These gel electrodes snap into second layer 6 Thus second layer 6 is provided with little contact buttons for each electrode.

FIG. 5A is side view of FIG. 4A. FIG. 5A is 3-dimensional showing of how the electrodes 20, 22, 24 and 26 are set into the patch body 72. The electrodes as snapped-in and are, in some embodiments, removable. This removal can occur between uses of the Breast Sense Feeding Monitor system, or when the measurement is finished. FIG. 5A is an illustration with the electrodes in place is what is applied to the breast prior to measurements being taken.

FIG. 5B shows what the patch looks like after the measurement is complete. The user would remove the patch body 72, throw away the disposable part of the electrode. For the next measurement, the user would attach a set of four new electrodes 20, 22, 24 and 26 to snaps 70. Snaps 70 that allow the securing of the disposable electrode into the patch body 72.

There are many variants on this design which will be apparent to an ordinary skilled artisan. By example, the four electrodes can be provided on one backing piece. This makes it easy to put them on.

An adhesive gel electrode, which is shown in FIG. 4C, is the traditional way of making contact to the body for impedance, EKG, or other electrical measurements. Typically gel electrodes provide a certain amount of impedance or resistance to current flow. This is in addition to the impedance provided by the skin and breast tissue. In order to perform a measurement, the impedance sensor circuit 8 and battery 16 must provide sufficient voltage and power to drive the desired current, typically between about 100 and 500 uA. The injected current must also be applied at a sufficiently high frequency, typically greater than about 10 kHz, so that a substantial part of the current can reach the interior of the breast through capacitive coupling.

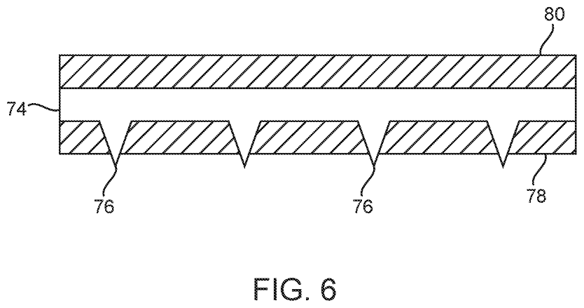

FIG. 6. illustrates employing a microneedle electrode 74 instead of a gel electrode in the Breast Sense Feeding Monitor system to optimize data collection.

In an alternative embodiment of the Breast Sense Feeding Monitor system shown in FIG. 6, the microneedle electrode 74 design is one where instead of a gel electrode, a microneedle electrode is used. The microneedle electrode has short, microneedles 76 that penetrates the skin slightly. Because the outer layer of the skin is very high resistance, better data is then obtained.

Microneedle electrode 74 has multiple microneedles 76, so there might be more than one. Microneedles 76 may be anywhere from 50 to 300 microns long. Microneedles 76 are typically made of stainless steel or silicon. Microneedle electrode 74 will still have adhesive layer 78 that would go either in between, or around the whole electrode. This adhesive layer 78 allows the microneedle electrode 74 to be applied and adhere to the skin. Also provided is conductive backing 80 for attaching a wire.

Microneedle electrode 74 may offer the advantage that they have a much lower resistance than the traditional electrodes. Microneedle electrode 74 multiple microneedles 76 are not deep enough to hit sub-dermal nerves. While thus not painful, microneedle electrode 74 may feel something like sandpaper to the user. As such microneedle electrodes 74 can have the disadvantage of being a bit uncomfortable to some users. However, this could be a good alternative for people who have a sensitivity to the adhesive.

Because the microneedle electrode 74 multiple microneedles 76 penetrate the dead layer of skin, they allow the current to be injected past that dead layer of skin. This means that the overall resistance to that current that is being injected by the drive electrodes is lowered. As a result, lower power is required, and a concomitantly smaller battery 16.

The battery 16 is a big part of the size of the patch. Employing microelectrode 74 in the design of the Breast Sense Feeding Monitor system can make the whole Breast Sense Feeding Monitor system device smaller and more comfortable to wear. This would be at the potential expense of local skin irritation.

Regarding the sense electrodes, because there is this dead layer of skin, the sense electrodes pick up the signal using “capacitive coupling”. The sense electrodes need to sense at several kilohertz to pick up that signal with the basic electrodes.

However, since electrodes with microneedles penetrate the dead skin, they actually make contact with the interstitial fluid just beneath that dead skin. As a result, the system can drive and sense at lower frequency, because the connection is ohmic. This is similar to the difference between connecting through a resistor versus through a capacitor. If the connection is through a capacitor, drive much happen at a high frequency. Thus use of microelectrode 74 has the potential to make the Breast Sense Feeding Monitor system circuits simpler, and lower power. This, in turn, would allow a smaller form factor for the Breast Sense Feeding Monitor system device.

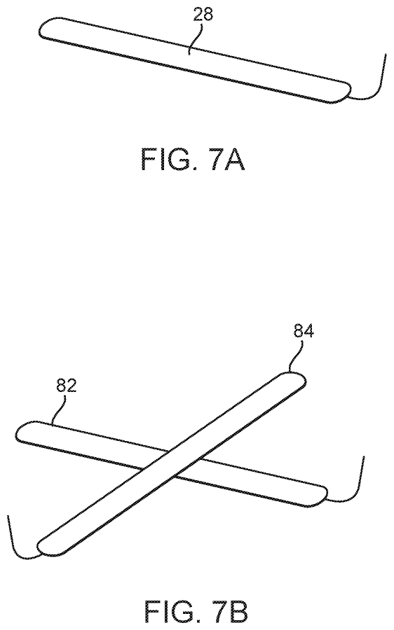

FIG. 7 shows various configurations for the strain gauge sensor 28. Strain gauge sensor 28 is typically a long piezoelectric sensor, whose resistance changes with how much it is bent. This is how strain gauge sensor 28 detects the curvature of the breast and movements like breathing, or deformation of the breast. The basic configuration is shown in FIG. 7A. In this case, strain gauge sensor 28 4-6 inches long and ¼ inch wide. As shown in the configuration of FIG. 7A strain gauge sensor 28 provides bending measurement in one direction. All these measurements can be varied to some degree.

Strain gauge sensor 28 can be in different patterns. As shown in FIG. 7B, two different strain gauges can be installed into the patch. While this design would make the Breast Sense Feeding Monitor device form factor larger, the design allows detection curvature and breast deformation in two dimensions.

Multiple piezoelectric strain gauges 82 and 84 can be provided in a cross pattern as shown in FIG. 7B. In other configurations, not shown in this view, piezoelectric strain gauges 82 and 84 would be provided back-to-back. This provides better measure of bend in both directions than the other direction.

The strain gauge connects to the strain sensor circuit 10 which is typically called a bridge circuit, in various manners well known to ordinary skilled artisans. By example, full bridges, half bridges, quarter bridges and other designs that translate that bending, and detecting the voltage or the resistance change that comes out of that.

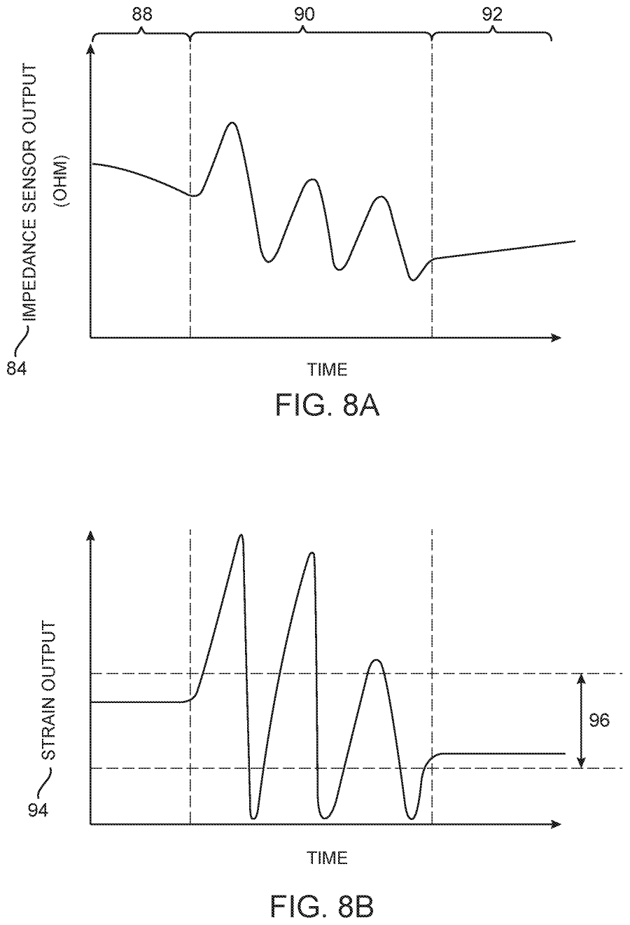

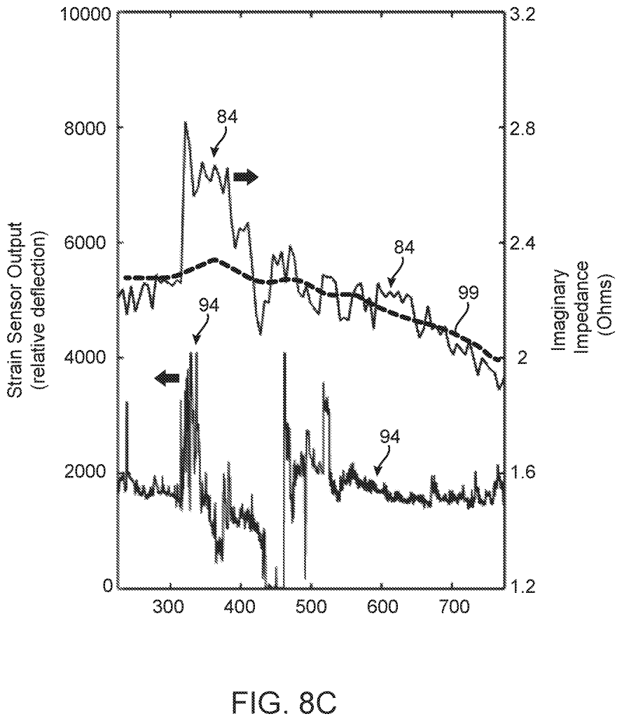

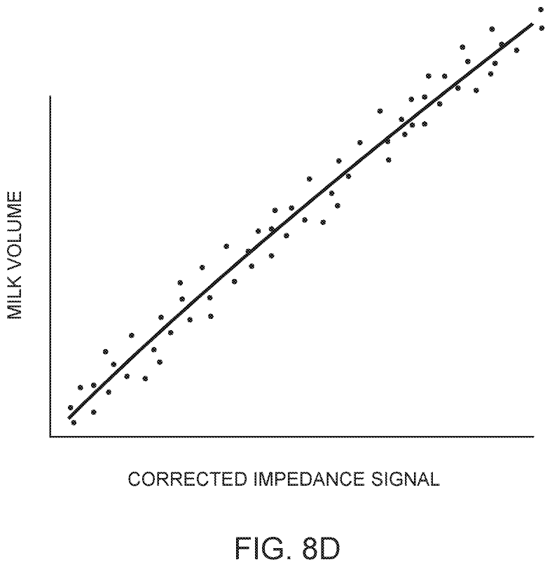

FIG. 8 illustrates how the two different sensors, strain and impedance, work in in conjunction with each other in the Breast Sense Feeding Monitor system to provide previously unavailable information on breast milk feeding. FIG. 8A shows the output of an impedance sensor for a typical feeding session.

There are several parameters that the impedance sensor outputs can provide, including the frequency and amplitude of the drive current and voltage, the amplitude of the time-varying detected at the sense electrodes, and the phase of the voltage at the sense electrodes relative to the drive current and voltage. These parameters are usually combined to report real and imaginary impedance values at each frequency. As known to those skilled in the art, amplitude and phase or real and imaginary values of the impedance are equivalent ways of referring to the same data output.

Additional parameters such as resistance, capacitance, or time constant values may be derived by fitting this data to theoretical models or equivalent circuits consisting of components such as resistors, capacitors, and constant phase elements. However, it is understood by those skilled in the art that biological impedance data can usually be fit to multiple theoretical models or equivalent circuits to obtain resistor and capacitor values. Therefore, resistance and capacitance values derived from the impedance sensor output are not necessarily unique. For this discussion, the impedance sensor output will be discussed in terms of the real and imaginary components of the impedance, but it is understood by those skilled in the art that resistance, capacitance, phase, and amplitude may offer equivalent ways of describing and analyzing the same data.

The base parameter, 86, shown in FIG. 8A is the imaginary component of the impedance sensor. The impedance sensor typically operates at one or more frequencies in the range of about 0.1 to 1 MHz, such as about 1 to 100 kHz In this basic configuration, two or three frequencies are used. This could be about 5 kHz, about 10 kHz, and about 20 kHz. At each of those frequencies the signal produces two numbers; a real and an imaginary value of impedance as is known to those skilled in the art.

FIG. 8A is the imaginary component of the detected impedance plotted at a particular frequency such as about 5 kHz versus time during a feeding. The data shows three distinct regions. Region 88 is the period before feeding starts, region 90 is the period during feeding, and region 92 is the period after the baby is finished feeding.

Before the feeding starts, the breast is full of milk. There is a baseline value of the imaginary component of the impedance. As the baby feeds, the imaginary component of the impedance drops to a final value at the end of feeding. During feeding, region 90, the change in the impedance signal, has a long-term change, a decline. This can be seen as the difference in the impedance signal plateaus in regions 88 and 92. However, because the impedance signal picks up deformations of the breast, there are typically undulations, or noise, associated with breathing, coughing, laughing, by the mother, the baby latching or detaching from the breast, swatting or grabbing the breast, or the mother compressing her breast to assist the baby in feeding.

All those deformations cause some kind of undulation in the breast, and that manifests itself as waves or wiggles in the detected impedance signal. The magnitude of these distortions during active feeding can be very substantial, up to 2 to 3× greater than the impedance change due to milk transfer out of the breast. Breast deformations may also account for some of the difference in impedance in the two plateaus in time regions 88 and 92, for example if the breast shape is different during time regions 88 and 92.

Without correcting for these substantial distortions, the system requires very careful operation to yield accurate data. For example, the mother must be in a consistent position and posture during the pre-feeding 88 and post-feeding periods 92 for about 2 to 5 minutes, without holding the baby or moving, in order to reliably measure the difference between the pre- and post-feeding plateaus in the impedance signal. This would cause significant inconvenience for the mother and baby and limit the system's ability to provide a real-time indication of the milk transferred to the infant during active feeding where most of the distortions occur.

The way the two sensors are used in the Breast Sense Feeding Monitor system, is that the impedance sensor is used as the main measurement, and the strain sensor is used to remove, that is correct for, some or most of these undulations that create noise during feeding, as shown later in the example of FIG. 8C.

FIG. 8B is an illustration of what the strain sensor output might look like during that same measurement. The strain sensor would show the shape or deformation of the breast. The strain sensor would also show waves that correspond to the big deformations seen in the impedance data.

Axis 94 is the output of the strain gauge sensor plotted over the same time regions pre-feed time region 88, during feed time region 90, and post-feed time region 92. Note that these time regions are common both to FIG. 8A and FIG. 8B. The way this output is used is twofold. One is that the output of the strain gauge can be used to derive a correction factor. For example, the correction actor may be the normalized value of the strain gauge signal, or a more complex function of the strain gauge output. The impedance signal is multiplied by this factor to correct for these deformations. When the impedance data in regions 88, 90, and 92 curve is multiplied by the correction factor, most of these undulations are removed. This operation may be performed by microprocessor unit in the Breast Sense Patch or the software on the mobile phone.

The second way to use the strain gauge data to advantage in the Breast Sense Feeding Monitor system is to define a band of strain gauge values that are considered the acceptable range of breast deformation that allows valid impedance data to be collected. Then, in the software, impedance data collected at time points when the strain sensor output is outside this band can be rejected or averaged with a lower weight factor than data collected during periods when the strain sensor is within the acceptable band. In other words, only utilize data when the breast is not severely distorted. If the breast is distorted too much, the data collected during that time is ignored. Basically, data collected during periods of distortion is considered invalid data.

This analysis distinguishes the native shape of the breast versus when it is subject to distortion, such as when the baby presses on it abruptly. If the baby presses, then deforms the breast so that data falls outside the acceptable band, the strain gauge informs the system that something is occurring, such as, the baby is compressing the breast, the mother mom moved or was like having a coughing fit, etc.

FIG. 8B, data band 96 is correlated with strain gauge output 94 to indicate that the shape of the breast is outside an acceptable range, and can be eliminated from the analysis. The only impedance data used are from time points where the strain sensor output is within this band 96. This eliminating a great deal of noise by eliminating the extremes where the breast is substantially deformed. As indicated above, this can be done during pre-feed time region 88, during feed time region 90, and post-feed time region 92 in FIG. 8A and FIG. 8B.

FIG. 8A shows the impedance data from the impedance sensor output, FIG. 8B shows the strain gauge output. The strain gauge output in two ways. First is to throw out things where the breast shape is substantially deformed. Second, for the things that are within the band, small changes in the breast shape can be corrected for by taking that strain data and using that as a factor to smooth out the impedance data. Having sensors of two different types allows, for the first time, real time measure of the milk transfer. Previously available methods were only able to ascertain the milk transfer, and the impedance signal, in the pre-versus post-nursing situations.

The impedance signal in preferred time region 88 and post-feed time region 92 provide a measure of milk transfer. However, for many mothers it is very useful to provide real-time measure of milk transfer during the feed time region 90. Many mothers want to be able to see in real time. The strain gauge component of the Breast Sense Feeding Monitor system enables that. It allows correction for all physical perturbations that happened during the feed which could confound the data. By example, during the pre- and the post-feeding periods, the mother could have a coughing fit that would be evident in regions 88 or 92, and could throw off the data. This unique capability of the Breast Sense Feeding Monitor system enables a lot better accuracy of the data.

Third thing that the two sensor types in the Breast Sense Feeding Monitor system enables is universal calibration. Because mothers have different breast shapes and sizes, prior systems rely on impedance require an individual calibration measurement to be done that involves having each mother feed a baby or breast pump or hand express a certain amount of milk, then inputting that milk volume, milliliters, into a computer. After that, these systems use that conversion factor to translate the impedance sensors' measurement to a volume of milk.

Unexpectedly, some of the present inventors have found that if the measurement is done carefully, it is possible to have a universal calibration curve even for a flexible patch such as the Breast Sense Patch 34. A universal calibration factor involves testing the system with a reference group of moms and babies, obtaining a calibration curve such as the one shown in FIG. 8D. This innovative conversion factor applies to all or most mothers. With this unique capacity, mothers do not have to perform an individual calibration in order to effectively use the Breast Sense Feeding Monitor system. The scatter in the calibration curve FIG. 8D is a factor of 2 better when the strain gauge correction is applied to the flexible patch than without.

The Breast Sense Feeding Monitor system strain gauge enables universal calibration for a flexible patch because it allows correction for differences in breast size and curvature. The strain sensor correction factor that allows the impedance signal to be normalized for different breast size or curvature. It may be desirable for clinical applications or for the highest accuracy to do both, to have a strain gauge, but also do an individual calibration with each mother. That gives the absolute best accuracy and precision.

This individualization procedure or method could involve a process similar to the following. When the mother puts on the patch, she initially hand expresses a certain amount of milk, by example, 2 ounces, that volume into a bottle. That volume is measured, and input into the mobile app. This becomes a calibration factor for translating the data to the best possible accuracy for an individual mom. However, for most application, there is sufficient accuracy in the Breast Sense Feeding Monitor system, to not have to do that, as there is correct for some of these noise factors, using that the two sensors, and other algorithm things, like smoothing, filtering, etc.

As shown in FIG. 8A, during the measurement the breast tends to make milk, even as the baby is feeding. As a result, there is oftentimes a slight slope to these pre- and post-measurements. This is observable in impedance data in region 88 and 92, seen as a slight slope to the measurement. That slope corresponds to the baseline production of milk in the breast. In the experience of some of the present inventors, this slope can be downward or upward. This depends on when the mother's milk start to be produced. This can be subtracted from the baseline, because typically the milk production is very slow. Being able to correct for the baseline production of milk is a unique capability of the Breast Sense Feeding Monitor system.

FIG. 9 demonstrates a further aspect of the Breast Sense Patch 34 that allows detection of additional feeding parameters such as sucks and swallows in addition to milk volume. If the Breast Sense Patch is close enough to the latch area on the breast, the impedance sensor output signal is also sensitive to distortions of the breast tissue causes by the sucking and swallowing movements in the infant's mouth. These distortions can have a characteristic pattern, as shown in FIG. 9B. The signal or y-axis in FIG. 9B is the real component of the impedance signal in ohms. The x-axis is time in seconds. The characteristic pattern for sucks and swallows consists of about 1 to 5 small amplitude waves (sucks) followed by a deep wave (swallow).

The detecting sensor must collect about 5 to 10 data points a second in order to detect sucks and swallows. In most infants, sucks occur approximately once every 0.8 to 1 second. Swallows occur approximately once every 1 to 5 seconds. In order to collect about 5 to 10 data points a second, the impedance sensor would run at a single frequency so that data at that frequency is collected and averaged every 100 msec or sooner. By comparison, for the detection of milk intake, the impedance sensor should be run at 2 or more frequencies, and data for each frequency should be average over 200 msec to 1 sec. This means that in multi-frequency mode, time points will be 1 to 5 seconds apart.

The inventors have discovered that what works best is to alternate between running the impedance measurement at a single frequency with running the impedance at multiple frequencies. FIG. 9 shows the frequency used for impedance measurement. By example, a single frequency, for example, 10 kHz, is run very quickly, so back to back 10 kHz measurements every 100 ms. This is the single frequency fast region 98. Then every 30 s-2 min two other frequencies are run, as in multi-frequency region 100. So for example, this could be done at 20 kHz and 5 kHz.

During, each of these, the Breast Sense Feeding Monitor system is constantly going back and forth between a single frequency region and multi-frequencies. Then a single frequency is run again, followed by multiple frequencies. The multiple frequencies are seen as the three parallel lines in the table. Single frequencies are one line.

Alternatively, multiple frequencies can be run during the pre-feed 88 and post-feed 92 time regions while a single frequency is run during the active feeding region 90. The reason a single frequency is run is that when during a single frequency reading, running a single frequency can collect data very quickly, always at the same frequency, it is possible, for the first time, to detect the baby's sucks and swallows. This innovative functionality is shown in FIG. 9B. FIG. 9A shows the operation of the Breast Sense Feeding Monitor system at different frequencies.

FIG. 9B shows showing detection of suck during the single frequency part of the measurement. During the single frequency part of the measurement, the impedance signal has very fine waves corresponding to sucks 102. These can be small rapid undulations, by example, that are superimposed on the larger signal. Then there are some dips that are swallows in between. After that the typical baby then goes into a patter on suck suck suck, and gulp, suck suck suck, and gulp. This is shown as swallows 104, and sucks 102.

The rapid, single frequency mode of operation allows detection of sucks and swallows, which has two benefits. One is, it actually allows a mother to assess the quality of the infant feding to help to optimize latch or how the baby is held. A baby that is well-latched will have a pattern of consecutive strings of sucks and swallows (for example, suck suck suck suck, swallow, suck suck suck suck suck, swallow . . . ) with few breaks. The baby that does not have a good latch or that is struggling with feeding due being premature or having neurological or motor problems will have irregular patterns of sucks and swallows, interspersed with periods where the baby detaches from the breast, cries, or takes a rest.

The second benefit of this fast measurement is that the sucks and swallows can be counted. Assuming a certain volume is swallowed, or is pulled for a typical suck, this is useful to error-check the standard impedance measurement. The result is more robust data. This provides a second way to calculate milk transfer. At least during that region, the average of the two can be taken. Alternatively, they can be combined in different ways.

For example, for infants younger than 2 or 3 days, milk production has not commenced or is too low to detect reliably through the standard impedance measurement (FIG. 8A). during this time, infants feed on the thick viscous fluid produced by the breast known as colostrum. Infants typically lose weight during this period, resulting in substantial anxiety among mothers as they weight for their milk to “come in”. During this period, it is possible to provide a measure of the progression towards onset of milk production (lactogenesis) by measuring and tracking the suck-to-swallow ratio and the number of suck-swallow bursts per minute. Providing these mothers with a measure of progression can substantially alleviate anxiety. It can also quickly identify mother-infant pairs where there may be a delay in lactogenesis and additional interventions such as supplementation or the use of a breast pump may be appropriate.

Alternative configurations of the Breast Sense Patch are possible that allow detection of the suck and swallow data with greater accuracy.

For example, in the Breast Sense patch configuration of FIG. 4A, one pair of electrodes may be used primarily for detecting sucks and swallows and a different pair for detecting milk volume. The different pairs may be connected to a single impedance sense circuit that switch between different pairs. Alternatively, they may have dedicated impedance sense circuits that allow simultaneous measurement of both pairs.

Sucks and swallows may also be detected by the strain sensor located inside the Breast Sense Patch, in place of or in addition to the impedance sensor. The strain sensor may superior better sensitivity. Both sensors may be used to detect sucks and swallows in order to cross check each other and eliminate artifacts.

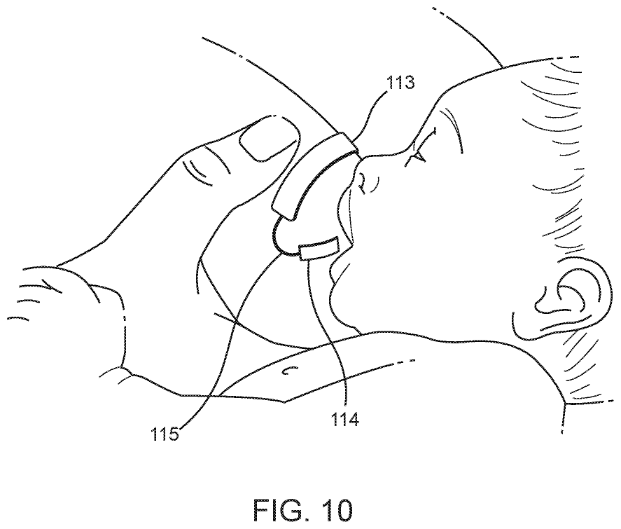

Alternatively, in FIG. 10, the sucks and swallows may be detected using 1 or more additional impedance sense electrodes or a dedicated strain sensor positioned inside or very close to the area where the baby latches onto the breast and connected to the Breast Sense patch via wires. While this location may not be ideal for detecting milk volume, it can provide higher sensitivity for detecting suck and swallow motions.

Alternatively, a second patch, called the Baby Patch 116, may be placed on the infant's throat, neck, or chin area that is specifically used for detecting sucks and swallows. This is shown in FIG. 11A. The Baby Patch may contain either an impedance sensor or strain sensor or both. It may attach to the baby's throat, chin, or cheek area, preferably underneath the chin where maximal displacements can be detected due to sucking and swallowing motions. It may connect wirelessly or via wires to the Breast Sense Patch 114. In some embodiments, the Baby Patch 116 sends it's digital signal wirelessly to the mobile phone 38. In other embodiments the Baby Patch may send it's signal wirelessly to the Breast Sense Patch 114 where the signals from the two patches can be combined to avoid latency issues that may arise if the signals are sent separately to a mobile phone that is running multiple software programs at the same time.

The Breast Sense Patch and the Baby Patch may be held in place using any suitable mechanism, including but not limited to the adhesive used in gel electrodes or other suitable hypoallergenic skin adhesive suitable for contact for the duration of multiple feedings.

### Example 1

FIG. 8C shows data from the Breast Sense Patch using impedance and strain sensor data. The impedance sensor output 84 was the imaginary component of the impedance at 50 kHz measured with a 6 inch Breast Sense Patch using gel electrodes (3M 2228 gel electrodes) and a piezoelectric strain sensor (4.5″ long piezoelectric, with a 2×10 kohm half bridge strain sensor circuit). The Breast Sense Patch was worn on the breast as depicted in FIG. 2A, at distance of 6 cm from the nipple. Impedance data was collected with a drive current of 500 uA at 5, 10, 20, 50, and 80 kHz during breastfeeding.

Line 84 was the imaginary component of the detected impedance at 50 kHz. This corresponds to the right axis in FIG. 8C. The strain sensor output 94 corresponds to the left axis and has arbitrary units. An upward deflection in the strain sensor indicates compression of the breast. At approximately 330 seconds from the start of feeding, the infant's movement caused a significant distortion of the breast from 2000 to 400 sensor bits.

This resulted in a corresponding step increase in the impedance signal from 2.2 ohm to 2.75 ohm, caused entirely by the deformation of the breast and does not indicate milk transfer. Another large change in breast shape occurs at approximately 450 seconds and is detected by deformation is detected by the strain sensor output 94. This resulted in a large downward change in the impedance curve 84.

After 500 seconds, the breast returned to an un-deformed state and the impedance data 84 is much less noisy. Using the strain sensor data to identify the regions of significant breast distortion and correct for the distortion allowed the impedance data to be corrected to obtain curve 99. The improvement in noise going from curve 84 to curve 99 could not be achieved simply by averaging or using the impedance data alone. Curve 99 was significantly less noisy and represents changes in impedance related to milk volume in the breast independent of breast deformation.

### Example 2

FIG. 11B shows a typical real time output of milk that was transferred, in mL of milk versus time, based on the Breast Sense Patch where the impedance signal was corrected with the strain gauge signal.

### Example 3

FIG. 11C is typical output from a Baby Patch, as would be located on a feeding infant's throat area. In this example, and using a strain gauge sensor output whose deflection indicate sucks 122 and swallows 120.

1-15. (canceled)

16. A breast sense feeding monitor system comprising:
a. two or more impedance sensor electrodes;
b. one or more breast shape sensors;
c. circuitry that receives signal from said impedance sensor electrodes and said breast shape sensor(s);
d. a microprocessor that receives signal from said circuitry;
e. a data transmitter;
f. an electrical power source; and
g. a user interface device which receives sensor data and provides information to a nursing mother.

17. The system of claim 16, wherein said impedance sensor electrodes are wet and/or dry.

18. The system of claim 16, where said signal from said impedance sensor electrodes is converted to milk volume data using a calibration function.

19. The system of claim 16, wherein one or more of said breast shape sensor(s) are strain sensors.

20. The system of claim 19, wherein said strain sensor(s) are capacitive, resistive, and/or piezoelectric.

21. The system of claim 16, wherein said signal from said breast shape sensor(s) is used to correct said signal from said impedance sensor electrodes for differences in breast sizes and shapes among individuals and/or over time.

22. The system of claim 16, wherein one or more of said sensors, said microprocessor, and/or said electrical power source are contained in a flexible structure.

23. The system of claim 16, wherein at least part of said system is disposable.

24. The system of claim 16, wherein said breast shape sensors improve said impedance sensor electrodes signal by about 10-200%, preferably by about 30-150%, and more preferably by about 100%.

25. The system of claim 16, wherein said impedance sensor electrodes signal is further improved with individual calibration.

26. The system of claim 16, wherein said user interface is a cell phone, a tablet, a computer screen, a smart watch, or a television screen.

27. The system of claim 26, wherein said user interface provides individualized calibration based on mapping of breast shape, size and/or curvature.

28. The system of claim 26, wherein said user interface provides display, sharing, tracking, analysis and/or storage of said data.

29. The system of claim 16, wherein said sensor data includes milk production, milk conveyance, milk content in the breast, colostrum content in the breast, baby sucking patterns, baby suck-swallow ratio, baby suck and swallow burst duration, baby swallow pattern times and rhythms, and/or sucking strength.

30. The system of claim 29, wherein said sensor data is compared to a predetermined reference data set.

31. The system of claim 29, wherein said sensor data is from multiple feeding sessions.

32. The system of claim 16, wherein said system collects said sensor data at a rate of about 5 to about 10 data points a second.

33. The system of claim 16, wherein automated biofeedback and/or lactation coaching is provided based on said data to a nursing mother through said user interface.

34. The system of claim 16, wherein lactation coaching is provided from a remote lactation consultant or other medical provider receiving said data to a nursing mother through said user interface.

35. The system of claim 16, wherein one or more of said sensors is positioned on the breast near the infant's latch area or on the infant's face or neck.

36. A baby sense feeding monitor system comprising:
a. one or more curvature sensors and/or at least two impedance sensor electrodes applied to an infant,
b. circuitry that receives signal from said curvature sensors and/or impedance sensor electrodes,
c. a microprocessor that receives signal from said circuitry,
d. a data transmitter,
e. an electrical power source.

37. A combined baby and breast sense feeding monitor system comprising the system of claim 1 and the system of claim 21.
